(19) United States
(12) Patent Application Publication
(10) Pub. No.: US 2022/0087545 A1
(43) Pub. Date: Mar. 24, 2022

(54) Pressure Relief Valve for Blood Pressure Cuff

(71) Applicant: Apple Inc., Cupertino, CA (US)

(72) Inventors: Pranay Jain, Mountain View, CA (US); Joseph R. Lee, Daly City, CA (US); William S. Lee, Fremont, CA (US); Zijing Zeng, San Jose, CA (US)

(21) Appl. No.: 17/398,775

(22) Filed: Aug. 10, 2021

Related U.S. Application Data
(60) Provisional application No. 63/081,668, filed on Sep. 22, 2020.

(51) Int. Cl. A61B 5/0235; A61B 5/021

(52) U.S. Cl. CPC A61B 5/0235; A61B 5/02141

(57) ABSTRACT

Embodiments are directed to a blood pressure measurement device that includes a cuff configured to wrap around a limb of a user when the cuff is worn by the user, a bladder coupled to the cuff and configured to compress the limb when inflated, and a pump operative to inflate the bladder. The blood pressure measurement device can also include a valve positioned between the bladder and the pump and configured to operate in a first state that allows air from the pump to enter the bladder and prevent air from leaving the bladder. The valve can also be configured to operate in a second state that allows air to leave the bladder. The blood pressure measurement device can also include a vent positioned between the bladder and an external environment and configured to continuously release air from the bladder.

### CROSS-REFERENCE TO RELATED APPLICATION

This application is a nonprovisional of, and claims the benefit under 35 U.S.C. § 119(e) of, U.S. Provisional Patent Application No. 63/081,668, filed Sep. 22, 2020, the contents of which are incorporated herein by reference as if fully disclosed herein.

### FIELD

The described embodiments relate generally to devices, systems, and methods for measuring a physiological parameter of a user. More particularly, the present embodiments relate to pressure relief valves for blood pressure measurement devices that are worn by a user.

### BACKGROUND

A user may monitor one or more of their physiological parameters by attaching a monitoring device such as a blood pressure monitor to one of their limbs. The blood pressure monitor may include a cuff that secures an inflatable bladder against a limb of the user. The bladder can be inflated to compress the limb, thereby compressing one or more blood vessels in the limb and restricting and/or stopping blood flow through the vessels. The various pressures in the inflated bladder that restrict and/or stop blood flow through the vessels in the limb may be measured and used to determine one or more physiological parameters of a user such as blood pressure of the user. After the blood pressure measurement is complete, the bladder can be deflated and may remain in this state until the next use.

In some cases, a pressure sensor in the cuff may be calibrated prior to a blood pressure measurement and while the cuff is deflated. However, residual pressure may remain in the bladder if it is not completely deflated, which may affect the accuracy of the calibration. In some cases, a user may continue to wear the blood pressure device between measurements. In these cases, residual pressure in the bladder can cause discomfort and/or restrict movement the user's limb. It may be desirable to remove or decrease residual pressure in the bladder.

### SUMMARY

Embodiments are directed to a blood pressure measurement device that includes a cuff configured to wrap around a limb of a user when the cuff is worn by the user, a bladder coupled to the cuff and configured to compress the limb when inflated, and a pump operative to inflate the bladder. The blood pressure measurement device can also include a valve positioned between the bladder and the pump and configured to operate in a first state that allows air from the pump to enter the bladder and prevent air from leaving the bladder, and also configured to operate in a second state that allows air to leave the bladder. The blood pressure measurement device can also include a vent positioned between the bladder and an external environment and configured to continuously release air from the bladder.

Embodiments are also directed to a blood pressure measurement device that includes a cuff configured to wrap around a limb of a user when the cuff is worn by the user, a bladder coupled to the cuff and configured to compress the limb when inflated, and a pump operative to inflate the bladder. The blood pressure measurement device can also include a valve that includes a housing and a diaphragm contained within the housing. The diaphragm can operate in a first state that allows the pump to inflate the bladder and operate in a second state that releases air from the bladder at a first rate. The blood pressure measurement device can also include a vent configured to continuously release air from the bladder at a second rate that is less than the first rate.

Embodiments are also directed to a blood pressure measurement device that includes a cuff configured to wrap around a limb of a user when the cuff is worn by the user, a bladder coupled to the cuff and configured to compress the limb when inflated, and a pump operative to inflate the bladder. The blood pressure measurement device can also include a valve that includes a housing defining an outlet that couples the bladder to an external environment and a diaphragm contained within the housing, where the diaphragm comprises an air permeable material. The diaphragm can operate in a first state that blocks the outlet and allows air to vent through the air permeable material and operate in a second state that allows air in the bladder to vent from the outlet.

### BRIEF DESCRIPTION OF THE DRAWINGS

The disclosure will be readily understood by the following detailed description in conjunction with the accompanying drawings, wherein like reference numerals designate like structural elements, and in which:

FIG. 1 shows an example of a blood pressure measurement device as worn by a user;

FIG. 2 shows an example of a blood pressure measurement device;

FIGS. 3A-3B show example configurations of blood pressure measurement devices;

FIGS. 4A-4B show example operations of blood pressure measurement devices;

FIGS. 5A-5C show an example blood pressure measurement device;

FIGS. 6A-6B show example vents that can be integrated with a blood pressure measurement device;

FIG. 7 shows an example blood pressure measurement device that includes an air permeable diaphragm; and

FIG. 8 shows a block diagram of a blood pressure measurement device.

It should be understood that the proportions and dimensions (either relative or absolute) of the various features and elements (and collections and groupings thereof) and the boundaries, separations, and positional relationships presented therebetween, are provided in the accompanying figures merely to facilitate an understanding of the various embodiments described herein and, accordingly, may not necessarily be presented or illustrated to scale, and are not intended to indicate any preference or requirement for an illustrated embodiment to the exclusion of embodiments described with reference thereto.

### DETAILED DESCRIPTION

Reference will now be made in detail to representative embodiments illustrated in the accompanying drawings. It should be understood that the following descriptions are not intended to limit the embodiments to one preferred embodiment. To the contrary, it is intended to cover alternatives, modifications, and equivalents as can be included within the spirit and scope of the described embodiments as defined by the appended claims.

Embodiments disclosed herein are directed to a blood pressure measurement device that includes a valve that is used to control inflation and deflation of a bladder, and a vent that operates to relieve residual pressure in the bladder. The blood pressure measurement device can include a cuff that wraps around the limb of a user and secures a bladder against the user's limb. The blood pressure measurement device can also include a pump that is used to inflate the bladder thereby compressing the user's limb to restrict blood flow through the limb. The blood pressure measurement device can include a valve that fluidly couples the pump to the bladder. As user herein the term “fluidly couple” may be used to refer to two or more volumes, elements structure, objects components, parts, or the like that are in fluid communication with one another such that fluid may flow between or among the two or more volumes, elements structure, objects components, parts, or the like. The valve can operate in various states. For example, when the bladder is being inflated, the valve can operate in a first state that allows the pump to inflate the bladder and prevent air from leaving the bladder. Accordingly, in the first state, the pump and valve operate to increase a pressure within the bladder. After the bladder has been completely inflated, the valve can operate in a second state that allows air contained within the bladder to leave the bladder. Typical valves may not allow the bladder to completely empty and, as a result, a residual pressure may remain in the bladder. This residual pressure may prevent pressure sensors from properly calibrating between blood pressure measurements. In some cases, the residual pressure can increase a stiffness of the cuff, thereby restricting the user's movements and/or restricting blood flow through the user's limb even when a blood pressure measurement is not being taken.

In some embodiments, the blood pressure measurement device can also include a vent that is fluidly coupled to the bladder. The vent can operate to continuously release air from the bladder and allow any residual pressure within the bladder to equalize with the surrounding environmental pressure. For example, the vent can be formed from an air permeable material, or other air permeable structure or component that fluidly couples the bladder to the external environment. The vent can be configured to slowly release air from the bladder such that an air release rate from the vent is much slower than the air release rate of the valve. In this regard, the valve may operate to quickly release air from the bladder and the vent may slowly release any residual pressure that is not released by the valve. The vent can also be configured to release air at a lower rate than the pump inflates the bladder. For example, a release rate of the vent may be much lower than an inflation rate of the pump such that the vent has a minimal effect on the pump inflating the bladder.

In some cases, the vent is integrated into the valve such as into a housing of the valve. The vent can include an air permeable material and one or more openings in the housing of the valve. For example, the vent can include an air permeable membrane such as a polytetrafluoroethylene (PTFE), open cell foam or other suitable material. In other cases, the vent can be one or more openings in the valve housing such as one or more small holes that fluidly couple the bladder to the external environment. In these cases, the size and number of holes may be configured to control a release rate of the vent. For example, the holes may be sized such that the release rate of air from the vent is lower than an inflation rate of the pump. In other embodiments, the vent can be incorporated into other portions of the blood pressure measurement device. For example, the vent can be integrated into the bladder to continuously release air from the bladder. In some cases, the valve can be a diaphragm valve and the vent can include one or more air permeable portions formed in the diaphragm of the valve.

For the purpose of explanation and clarity, the blood pressure measurement device is described as being operated by using air to inflate and deflate the bladder. However, other fluids can be used in place of air. The fluids can include substances that have no fixed shape, which allows them to flow, including air, other gasses, liquids, or combinations thereof.

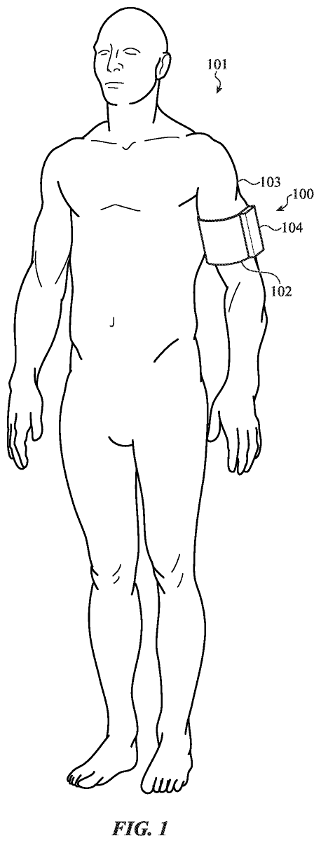

These and other embodiments are discussed below with reference to FIGS. 1-8. However, those skilled in the art will readily appreciate that the detailed description given herein with respect to these Figures is for explanatory purposes only and should not be construed as limiting.

FIG. 1 shows an example of a blood pressure measurement device 100 as worn by a user 101. The blood pressure measurement device 100 can include a cuff 102 that wraps around a limb 103 of a user 101. The cuff 102 can include a bladder that is operative to expand inward to compress the limb 103 thereby restricting blood flow through one or more vessels in the limb 103. The blood pressure measurement device 100 can also include a housing 104 that is attached to the cuff 102.

The cuff 102 can form an elongated flexible sheet that extends from a first end portion to a second end portion. The cuff 102 can be wrapped around the limb 103 and the first and second end portions can be coupled by a fastening mechanism, which can include any suitable mechanism that secures the first and second ends of the cuff 102, examples of which can include hook-and-loop fasteners, clips, zippers, buttons, and so on. In some cases, the first and second end portions can be permanently joined and the cuff 102 can be placed on the limb 103 by a user 101 sliding a limb 103 through a central opening defined by the cuff 102. In some cases, the cuff 102 can be substantially inextensible such that the cuff 102 remains approximately the same size when the bladder is inflated around the limb 103. Accordingly, inflating the bladder causes the bladder to expand inward thereby compressing the limb 103.

The housing 104 can contain components that are used to take a blood pressure measurement. In some embodiments, the housing 104 can include an air pump for inflating the bladder and positioned along at least a portion of the interior of the cuff 102. The housing 104 can further contain one or more components for operating the blood pressure measurement device 100, such as a pressure sensor that is used to measure a pressure within the bladder, the pump, one or more valves, a processor, memory, display, power source, and so on.

The blood pressure measurement device 100 can be operated to take a blood pressure of the user 101 by using a sensor to detect changes in blood flow due to collapse of blood vessels caused by compression of the limb 103. An air pump can be operated to inflate the bladder. As the bladder inflates, the sensor can be operated to detect changes in blood flow through blood vessels in the limb 103 and output a first signal that is indicative of the blood flow. A pressure sensor can also be operated to measure pressure within the bladder and output a second signal that is indicative of the pressure within the bladder. The first signal can be processed, such as by filtering and/or performing analysis, to identify sounds in the blood vessel that result from expanding the bladder and compressing blood vessels within the limb 103. For example, a first sound can be identified in the sensor signal that corresponds to the blood vessel beginning to collapse from the pressure applied by the bladder. In some cases, this first sound may correspond to a first Korotkoff sound. The air pressure in the bladder corresponding to the first sound can be determined from the air pressure sensor signal and used to estimate the diastolic blood pressure of the user 101. The air pressure in the bladder can continue to be increased and a second sound that occurs just prior to the stoppage of blood flow through the vessel can be identified from the sensor signal. In some cases, this second sound can correspond to a second Korotkoff sound. The air pressure in the bladder corresponding to the second sound can be determined from the air pressure sensor signal and used to estimate a systolic blood pressure of the user 101.

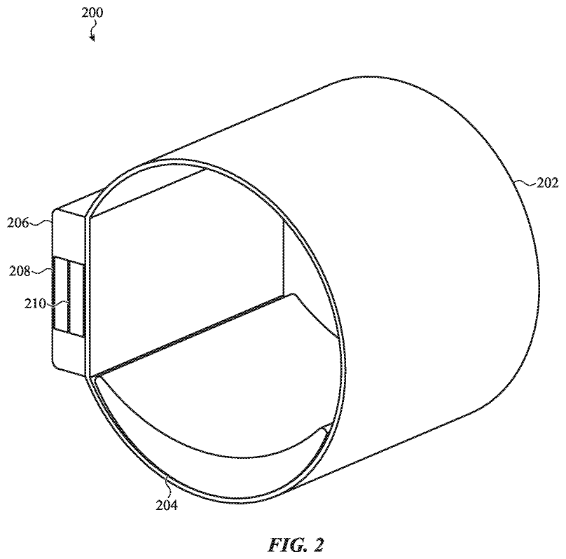

FIG. 2 shows an example a blood pressure measurement device 200. The blood pressure measurement device 200 can include a cuff 202 having a bladder 204, and a housing 206, and may be an example of the blood pressure measurement devices described herein. The housing 206 can include a pump 208 and a valve 210.

The pump 208 can be fluidly coupled to the bladder 204 and configured to inflate the bladder 204 by pumping air or other fluid into the bladder 204. The valve 210 can fluidly couple the pump 208 to the bladder 204. During inflation of the bladder 204, the valve 210 can operate in a first state that allows air from the pump to flow into the bladder and prevents air from exiting the bladder. For example, the valve 204 can prevent air in the bladder from flowing back into the pump and/or block one or more outlets to prevent air within the bladder from flowing through the outlet. In this regard, in the bladder 204 can be inflated such that the air pressure inside the bladder 204 increases. After the bladder 204 has been inflated by a desired amount, such as to a desired internal pressure, the valve 210 can transition to a second state that allows air to release from the bladder 204, thereby decreasing the air pressure within the bladder 204. In the second state, the valve 210 can allow air to escape from one or more outlets that fluidly couple the bladder 204 to the external environment. For example, in the second state, the valve 210 may transition, such that it no longer blocks an outlet from the bladder 204.

In some embodiments, the valve 210 can be a passive valve that transitions between the first and second states in response to one or more external factors such as operation of the pump. For example, the valve 210 can be a diaphragm valve, and when the pump is operated to inflate the bladder 204, a first portion of the diaphragm opens to allow air from the pump 208 to flow into the bladder 204. When the pump 208 is turned off and no longer pumping air into the bladder 204, the pressure inside the bladder 204 can cause the diaphragm to transition to a second state that opens one or more outlets and allows air to escape from the bladder 204. Other examples of passive valves can include check valves, or the like. In other cases, the valve 210 can include active valves, which can be electronically or pneumatically controlled to transition between a first state and a second state. In some cases, the active valves can be controlled by a processor or other control hardware contained within the housing 206. Examples of active valves include valves that are actuated by a solenoid or other suitable components. The valves 210 can include ball valves, needle valves, butterfly valves, pinch valves, or other suitable valves.

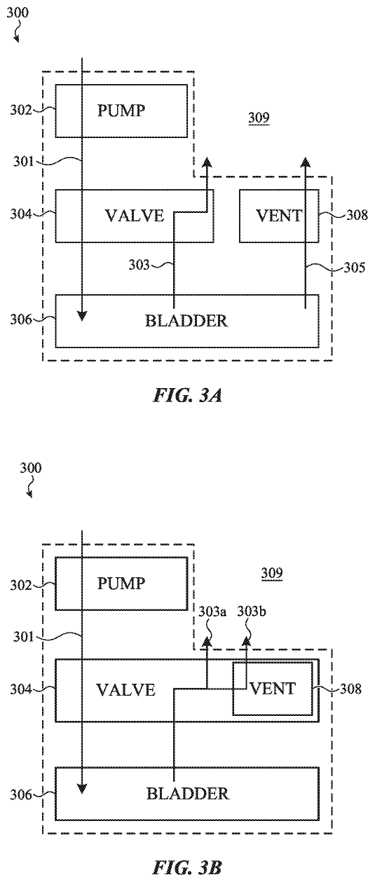

FIG. 3A shows an example configuration of a blood pressure measurement device 300. The blood pressure measurement device 300 can include a pump 302, a valve 304, a bladder 306, and a vent 308, which may be examples of similar components described here. The pump 302 can be connected to the bladder 306 by the valve 304 to create a first fluid path 301 that is used to inflate the bladder 306. The valve 304 can also fluidly couple the bladder 306 to the external environment 309 to create a second fluid path 303 that can be used to deflate the bladder 306. The blood pressure measurement device 300 can also include a vent 308 that connects the bladder 306 to the external environment 309 to create a third fluid path 305 that provides a continual release of air from the bladder 306.

In some embodiments, the pump 302 can be configured to inflate the bladder 306 with air from the external environment 309 via the first fluid path 301. The pump 302 can be an ultrasonic pump, diaphragm pump, or other suitable pump that is used to increase the air pressure within the bladder 306. The pump 302 can be coupled to the valve 304 and bladder 306 by one or more fluid impermeable conduits, such as pneumatic tubing that defines the first fluid path 301. In some cases, the pump 302 and the valve 304 can be contained within a housing that is attached to the bladder 306 as described herein. The valve 304 can also be configured to deflate the bladder 306 by releasing air from the bladder 306 to the external environment 309 via the second fluid path 303. The valve 304 can be used to selectively control the release of air from the bladder 306. For example, the valve 304 can be operative to transition between different states that selectively open and close the first and second fluid paths 301 and 303 to control air flow into and out of the bladder 306. In some embodiments, the valve 304 is controlled such that it only releases air from the bladder 306 in response to an external event or control signal. For example, when the valve 304 is a passive valve, it may only release air from the bladder 306 when the pump is turned off such that the air pressure of the pump 302 on the valve 304 drops below an air pressure of the bladder 306. In other examples, when the valve 304 is an active valve, it may only release air from the bladder 306 when it receives a signal from a controller to open a release port.

The vent 308 can connect the bladder 306 to the external environment 309 to create a third fluid path 305 that allows an air pressure within the bladder 306 to equalize with an air pressure of the external environment 309. The vent 308 can create a non-selective fluid path that allows air to continually move between the bladder 306 and the external environment 309. In this regard, as the bladder 306 is being inflated by the pump 302, the vent 308 may allow a portion of the air pumped into the bladder 306 to release into the external environment 309. The vent 308 can be configured to have an air release rate that is lower than an air inflation rate of the pump 302 pumping air into the bladder 306. For example, the air release rate of the vent 308 may be very small such that it has a minimal detrimental effect on the pump 302 inflating the bladder 306. When the pump 302 is turned off, the vent 308 allows a slow release of air from the bladder 306. The release of air by the vent 308 may be lower than an air release rate from the valve 304. In this regard, air can be selectively released from the bladder 306 by the valve 304 and continually released from the bladder 306 by the vent 308.

In some embodiments, the vent 308 can be formed from an air permeable material. For example, the vent 308 can be formed from a PTFE material, open cell foam, an air filter such as a metal air filter, or other suitable air permeable material. In other embodiments, the vent 308 can be created by forming one or more openings in the bladder 306 such as small holes that allow a slow release of air from the bladder 306. As illustrated in FIG. 3A, the vent 308 can be an independent component that is separate from the valve 304. For example, the vent 308 can be formed in a wall of the bladder 306 or directly coupled to the bladder 306.

As illustrated in FIG. 3B, in some embodiments, the blood pressure measurement device 300 can include a vent 308 that is integrated with the valve 304. For example, the vent can be formed in or coupled to the valve housing. In this regard, the valve 304 and the vent 308 may share at least a portion of the second fluid path 303 from the bladder 306 to the external environment 309. In some cases, the second fluid path 303 may include a first sub path 303a where the valve 304 selectively releases air from the bladder 306 to the external environment 309. The second fluid path 303 can also include a second sub path 303b where the vent 308 continually allows air pressure inside the bladder 306 to equalize with the external environment 309.

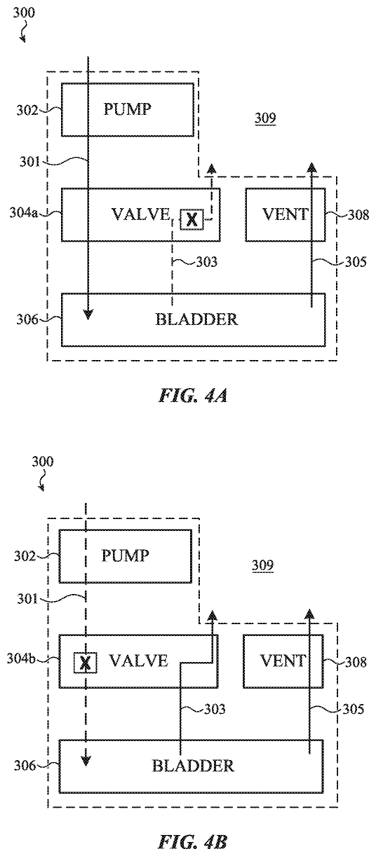

FIGS. 4A and 4B show example operations of a blood pressure measurement device 300, as described herein. FIG. 4A shows the valve 304a in a first state. In the first state, the valve 304a allows air from the pump 302 to enter the bladder 306 and prevents air from leaving the bladder 306. In this regard, the pump 302 can be operated to pump air into the bladder 306 via the first fluid path 301 thereby increasing an air pressure within the bladder 306. In the first state, the second fluid path 303 is closed thereby preventing air from leaving the bladder 306 via the valve 304a. In the first state, the vent 308 continually releases air from the bladder 306 via the third fluid path 305. As described herein, the air release rate of the vent 308 can be configured to be lower than the inflation rate of the pump 302. Accordingly, when the valve 304a is in the first state, the pump 302 can be operated to inflate the bladder 306 thereby increasing the air pressure within the bladder 306.

FIG. 4B shows the valve 304b in a second state. In the second state, the valve 304b allows air to release from the bladder 306 and to the external environment 309 via the second fluid path 303. The valve 304b may also prevent air from flowing from the bladder 306 and back to the pump 302. In the second state, the vent 308 continually releases air from the bladder 306 via the third fluid path 305. The release rate of air from the valve 304b may be greater than the release rate of air from the vent 308. Accordingly, when the valve 304b is in the second state, air is released from the bladder 306 via both the valve 304b and the vent 308. In some cases, the valve 304 may not be operative to completely equalize the air pressure within the bladder 306 with the air pressure of the external environment 309. For example, once the air pressure in the bladder 306 falls below a certain level, the second fluid path 303 through the valve 304b may shut leaving a small residual pressure in the bladder 306. The vent 308 may continue to release air from the bladder 306 via the third fluid path 305 thereby allowing the air pressure within the bladder 306 to equalize with an air pressure of the external environment 309. The vent 308 may continue to allow an air pressure within the bladder 306 to equalize with an air pressure of the external environment 309, for example as temperature changes or other factors cause air pressure differences between the bladder 306 and the external environment 309. In this regard, the vent 308 may allow more accurate calibration of pressure sensors that sense a pressure within the bladder 306 and/or allow the bladder 306 to remain flexible and compliant around a user's limb.

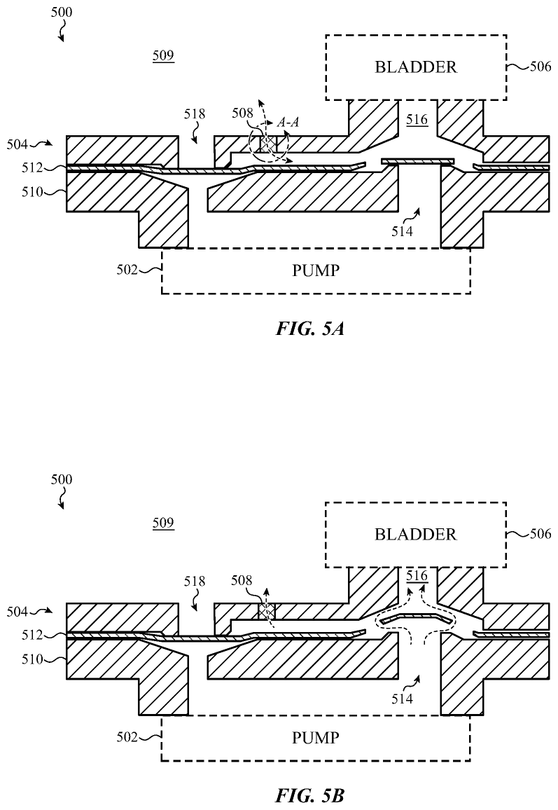

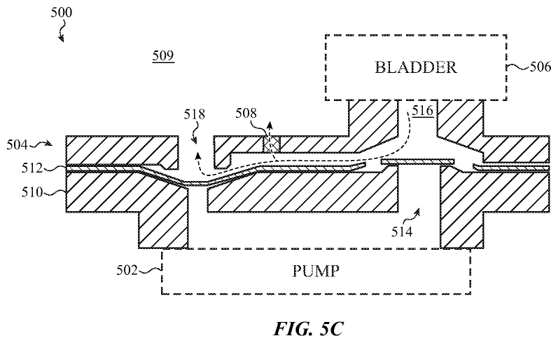

FIGS. 5A-5C shows an example blood pressure measurement device 500 that includes a valve 504 in various operational states. The valve 504 is shown in cross-section to illustrate the internal components of the valve 504. The blood pressure measurement device 500 can be an example of the blood pressure measurement devices described herein, and can include a pump 502, the valve 504, and a bladder 506. The valve 504 can be an example of a diaphragm valve that includes a housing 510 and a diaphragm 512 that define an inlet 514 from the pump 502, a first outlet 516 to the bladder 506, and a second outlet 518 to the external environment 509. The valve 504 can also include a vent 508, which can be an example of the vents described herein.

FIG. 5A shows the valve 504 in a neutral configuration when air is not being pumped from the pump 502 to the bladder 506 and air is not flowing from the bladder 506 and to the external environment 509. In the neutral configuration, the diaphragm 512 covers the inlet 514 to block air movement between the pump 502 and the bladder 506. The diaphragm 512 can be formed from a compliant material that is impermeable to air. For example, the diaphragm 512 can be formed from a rubber, silicone, urethane, or any other suitable material. In the neutral configuration, the pump 502 can be turned off such that it is not pumping air into the inlet 514 and does not exert pressure on the diaphragm 512. Also, in the neutral configuration, an air pressure within the bladder 506 is substantially equalized with an air pressure of the external environment via the vent 508. Accordingly, no air pressure is being exerted on the diaphragm 512 via the bladder 506.

FIG. 5B shows an example of the valve 504 in a first state when the pump 502 is being operated to inflate the bladder 506. Operation of the pump 502 can cause the air pressure in the inlet 514 to increase thereby deforming the diaphragm 512 to create an opening between the diaphragm 514 and the housing 510. The opening allows air from the pump 502 to flow into the bladder 506. Also, in the first state, the diaphragm 512 blocks the second outlet 518 due to air pressure from the pump 502. Accordingly, air within the bladder 506 cannot release from the valve 504 via the second outlet 518. In the first state, the vent 508 allows air a continual release of higher pressure air from the pump 502 and/or bladder 506 to the external environment 509. Accordingly, as the pump 502 is pumping air into the bladder 506, the vent 508 releases a portion of that air into the external environment 509.

FIG. 5C shows an example of the valve 504 in a second state when the pump 502 is turned off and the valve 504 is venting air from the bladder 506 to the external environment 509. After the pump has been used to inflate the bladder 506 and is turned off, the air pressure in the bladder 506 exceeds the air pressure of the inlet 514, which causes the diaphragm 512 to close over the inlet 514, thereby blocking the air path from the bladder 506 to the pump 502. The higher air pressure in the bladder 506 causes the diaphragm 512 to deform at the second outlet 518, thereby unblocking the second outlet 518 and allowing air to release from the bladder 506 and into the external environment 509. In the second state, the vent 508 allows air a continual release of higher pressure air from the bladder 506 to the external environment 509. Accordingly, air is released from both the second outlet 518 and the vent 508. As the air pressure within the bladder 506 decreases, elastic energy stored within the diaphragm 512 due to the deformation of the diaphragm 512 can cause the diaphragm 512 to block the outlet 518 before the air pressure within the bladder 506 has equalized with the air pressure of the external environment 509. Accordingly, a residual pressure may remain in the bladder 506, which is released by the vent 508.

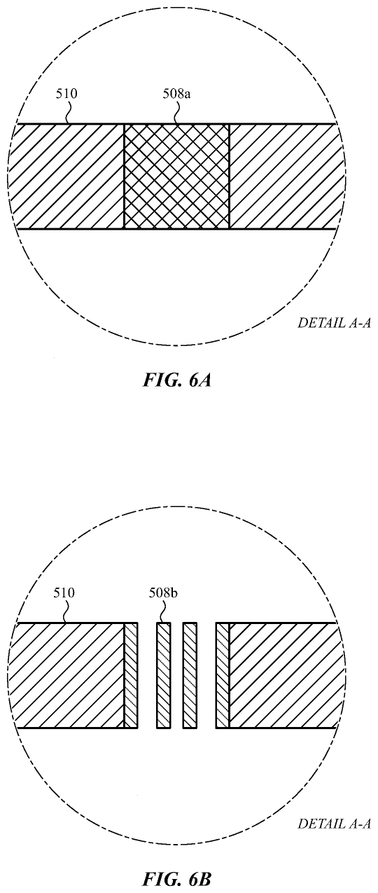

FIGS. 6A and 6B show example vents that can be integrated with the blood pressure measurement devices described herein. FIGS. 6A and 6B show detailed views of detail A from FIG. 5A as alternative examples of vents that can be integrated into the housing 510 of the valve 504. In the example of FIG. 6A, the vent 508a can include an air permeable material that is coupled to the housing 510. Examples of air permeable materials can include PTFE, open cell foams, filter elements such as paper, polymer or metallic filters, expanded metals, and so on. In some cases, the vent 508a can be an air permeable membrane that is coupled to the housing 510. In the example of the FIG. 6B, the vent 508b can be include one or more holes formed in the wall of the housing 510. The size of the holes can be configured to control the flow rate of air into or out of the housing 510. For example, the size and/or number of holes in the vent 508b can be configured to release air at a lower rate than an inflation rate of the pump as described herein.

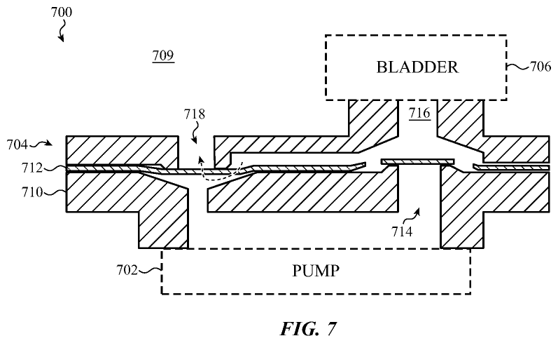

FIG. 7 shows an example blood pressure measurement device 700 with the valve 704 shown in cross-section to illustrate the internal components of the valve 704. The blood pressure measurement device 700 can be an example of the blood pressure measurement devices described herein, and can include a pump 702, the valve 704, and a bladder 706. The valve 704 can be an example of a diaphragm valve that includes a housing 710 and a diaphragm 712 that define an inlet 714 from the pump 702, a first outlet 716 to the bladder 706, and a second outlet 718 to the external environment 709. In the example shown in FIG. 7, the diaphragm 712 includes an air permeable material that allows air to pass through one or more portions of the diaphragm 712. In this regard, the diaphragm 712 can also function as the vent, as described herein.

In some embodiments, the entire diaphragm 712 is formed from an air permeable material such as PTFE. In these cases, air can pass through the diaphragm 712 to equalize the pressure between the bladder 706 and the external environment 709. In other cases, the one or more portions of the diaphragm 712 can be air permeable while other portions of the diaphragm 712 are air impermeable. In these cases, the size, location, and thickness of the air permeable sections can be configured to provide desired venting properties through the diaphragm 712.

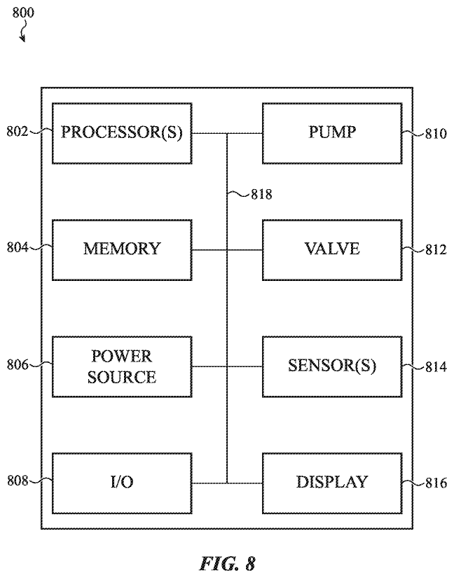

FIG. 8 shows a block diagram of a blood pressure measurement device 800, which may in some cases take the form of any of the blood pressure measurement devices or components thereof described with reference to FIGS. 1-8. The blood pressure measurement device 800 can include a processor 802, memory 804, a power source 806, an input/output (I/O) mechanism (or device) 808, a pump 810, one or more valves 812, one or more sensors 814, and a display 816.

The processor 802 can control some of all of the operations of the blood pressure measurement device 800. The processor 802 can communicate, either directly or indirectly, with some or all of the components of the electronic device 800. For example, a system bus or other communication mechanism 818 can provide communication between the processor 802, the memory 804, the power source 806, the input/output (I/O) mechanism 808, the pump 810, one or more valves 812, one or more sensors 814, and the display 816.

The processor 802 can be implemented as any electronic device capable of processing, receiving, or transmitting data or instructions. For example, the processor 802 can be a microprocessor, a central processing unit (CPU), an application-specific integrated circuit (ASIC), a digital signal processor (DSP), or combinations of such devices. As described herein, the term “processor” is meant to encompass a single processor or processing unit, multiple processors, multiple processing units, or other suitable computing element or elements.

It should be noted that the components of the blood pressure measurement device 800 can be controlled by multiple processors. For example, select components of the blood pressure measurement device 800 (e.g., a sensor 814) may be controlled by a first processor and other components of the blood pressure measurement device 800 (e.g., the display 816) may be controlled by a second processor, where the first and second processors may or may not be in communication with each other.

The memory 804 can store electronic data that can be used by the blood pressure measurement device 800. For example, the memory 804 can store electrical data or content such as, for example, audio and video files, documents and applications, device settings and user preferences, timing signals, control signals, and data structures or databases. The memory 804 can be configured as any type of memory. By way of example only, the memory 804 can be implemented as random access memory, read-only memory, Flash memory, removable memory, other types of storage elements, or combinations of such devices.

The power source 806 can be implemented with any device capable of providing energy to the blood pressure measurement device 800. For example, the power source 806 may be one or more batteries or rechargeable batteries. Additionally or alternatively, the power source 806 can be a power connector or power cord that connects the blood pressure measurement device 800 to another power source, such as a wall outlet.

The I/O mechanism 808 can transmit and/or receive data from a user or another electronic device. An I/O mechanism 808 can include a display, a touch sensing input surface, one or more buttons (e.g., a graphical user interface “home” button), one or more cameras, one or more microphones or speakers, one or more ports, such as a microphone port, and/or a keyboard. Additionally or alternatively, an I/O device or port can transmit electronic signals via a communications network, such as a wireless and/or wired network connection. Examples of wireless and wired network connections include, but are not limited to, cellular, Wi-Fi, Bluetooth, IR, and Ethernet connections.

The blood pressure measurement device 800 can also include a pump 810 that is used to inflate the bladder. The pump 810 can be an air pump such as an ultrasonic air pump, diaphragm pump, and so on. The pump 810 can be powered by the power source 806 and controlled by the processor 802. The pump 810 can be capable of inflating the bladder to pressures that are above typical human blood pressures such as up to or higher than 180 mm Hg. In some cases, the pump 810 can be configured to apply a linear or smooth pressure ramp within the bladder. The pump 810 can also be configured to provide a linear, or constant, pressure release within the bladder, which may be used to acquire blood pressure measurements during deflation of the cuff.

The blood pressure measurement device 800 can also include one or more valves 812 that fluidly couple the pump 810 to a bladder. The valves 812 can be passive valves, such as diaphragm valves described herein. In some cases, the valves 812 can be electronically controlled valves such as a solenoid actuated valve. In this regard, the electronically controlled valve can be electrically coupled to the processor 802 such that operation of the valve 812 is controlled by the processor 802.

The blood pressure measurement device 800 may also include one or more sensors 814 positioned almost anywhere on the blood pressure measurement device 800. The sensor(s) 814 can be configured to sense one or more type of parameters, such as but not limited to, pressure, sound, light, touch, heat, movement, relative motion, biometric data (e.g., biological parameters), and so on. For example, the sensor(s) 814 may include a pressure sensor, an auditory sensor, a heat sensor, a position sensor, a light or optical sensor, an accelerometer, a pressure transducer, a gyroscope, a magnetometer, a health monitoring sensor, and so on. Additionally, the one or more sensors 814 can utilize any suitable sensing technology, including, but not limited to, capacitive, ultrasonic, resistive, optical, ultrasound, piezoelectric, and thermal sensing technology.

The blood pressure measurement device 800 may also include a display, a display 816. The display 816 may include a liquid-crystal display (LCD), organic light emitting diode (OLED) display, light emitting diode (LED) display, or the like. If the display 816 is an LCD, the display 816 may also include a backlight component that can be controlled to provide variable levels of display brightness. If the display 816 is an OLED or LED type display, the brightness of the display 816 may be controlled by modifying the electrical signals that are provided to display elements. The display 816 may correspond to any of the displays shown or described herein.

As described above, one aspect of the present technology relates to measuring physiological parameters of a user. The present disclosure contemplates that in some instances this gathered data may include personal information data that uniquely identifies or can be used to contact or locate a specific person. Such personal information data can include demographic data, location-based data, telephone numbers, email addresses, twitter IDs (or other social media aliases or handles), home addresses, data or records relating to a user's health or level of fitness (e.g., vital signs measurements, medication information, exercise information), date of birth, or any other identifying or personal information.

The present disclosure recognizes that the use of such personal information data, in the present technology, can be used to the benefit of users. For example, the personal information data can be used to provide haptic or audiovisual outputs that are tailored to the user. Further, other uses for personal information data that benefit the user are also contemplated by the present disclosure. For instance, health and fitness data may be used to provide insights into a user's general wellness, or may be used as positive feedback to individuals using technology to pursue wellness goals.

The present disclosure contemplates that the entities responsible for the collection, analysis, disclosure, transfer, storage, or other use of such personal information data will comply with well-established privacy policies and/or privacy practices. In particular, such entities should implement and consistently use privacy policies and practices that are generally recognized as meeting or exceeding industry or governmental requirements for maintaining personal information data private and secure. Such policies should be easily accessible by users, and should be updated as the collection and/or use of data changes. Personal information from users should be collected for legitimate and reasonable uses of the entity and not shared or sold outside of those legitimate uses. Further, such collection/sharing should occur after receiving the informed consent of the users. Additionally, such entities should consider taking any needed steps for safeguarding and securing access to such personal information data and ensuring that others with access to the personal information data adhere to their privacy policies and procedures. Further, such entities can subject themselves to evaluation by third parties to certify their adherence to widely accepted privacy policies and practices. In addition, policies and practices should be adapted for the particular types of personal information data being collected and/or accessed and adapted to applicable laws and standards, including jurisdiction-specific considerations. For instance, in the US, collection of or access to certain health data may be governed by federal and/or state laws, such as the Health Insurance Portability and Accountability Act (“HIPAA”); whereas health data in other countries may be subject to other regulations and policies and should be handled accordingly. Hence different privacy practices should be maintained for different personal data types in each country.

Despite the foregoing, the present disclosure also contemplates embodiments in which users selectively block the use of, or access to, personal information data. That is, the present disclosure contemplates that hardware and/or software elements can be provided to prevent or block access to such personal information data. For example, in the case of determining spatial parameters, the present technology can be configured to allow users to select to “opt in” or “opt out” of participation in the collection of personal information data during registration for services or anytime thereafter. In addition to providing “opt in” and “opt out” options, the present disclosure contemplates providing notifications relating to the access or use of personal information. For instance, a user may be notified upon downloading an app that their personal information data will be accessed and then reminded again just before personal information data is accessed by the app.

Moreover, it is the intent of the present disclosure that personal information data should be managed and handled in a way to minimize risks of unintentional or unauthorized access or use. Risk can be minimized by limiting the collection of data and deleting data once it is no longer needed. In addition, and when applicable, including in certain health related applications, data de-identification can be used to protect a user's privacy. De-identification may be facilitated, when appropriate, by removing specific identifiers (e.g., date of birth, etc.), controlling the amount or specificity of data stored (e.g., collecting location data at a city level rather than at an address level), controlling how data is stored (e.g., aggregating data across users), and/or other methods.

Therefore, although the present disclosure broadly covers use of personal information data to implement one or more various disclosed embodiments, the present disclosure also contemplates that the various embodiments can also be implemented without the need for accessing such personal information data. That is, the various embodiments of the present technology are not rendered inoperable due to the lack of all or a portion of such personal information data. For example, haptic outputs may be provided based on non-personal information data or a bare minimum amount of personal information, such as events or states at the device associated with a user, other non-personal information, or publicly available information.

The foregoing description, for purposes of explanation, used specific nomenclature to provide a thorough understanding of the described embodiments. However, it will be apparent to one skilled in the art that the specific details are not required in order to practice the described embodiments. Thus, the foregoing descriptions of the specific embodiments described herein are presented for purposes of illustration and description. They are not targeted to be exhaustive or to limit the embodiments to the precise forms disclosed. It will be apparent to one of ordinary skill in the art that many modifications and variations are possible in view of the above teachings.

What is claimed is:

1. A blood pressure measurement device comprising:
a cuff configured to wrap around a limb of a user;
a bladder coupled to the cuff and configured to compress the limb when inflated;
a pump operative to inflate the bladder;
a valve positioned between the bladder and the pump and operative to:
in a first state, allow air from the pump to enter the bladder and prevent the air from leaving the bladder; and
in a second state, allow air to leave the bladder; and
a vent positioned between the bladder and an external environment and configured to continuously release air from the bladder.

2. The blood pressure measurement device of claim 1, wherein:
the valve comprises:
a housing defining an inlet coupled to the pump and an outlet that vents to the external environment; and
a diaphragm positioned between the inlet and the outlet;
in the first state, the diaphragm allows air to flow through the inlet and blocks air flow through the outlet;
in the second state, the diaphragm blocks the air flow through the inlet and allows the air to flow through the outlet; and
the vent comprises an air permeable membrane.

3. The blood pressure measurement device of claim 1, wherein a release rate of the air through the vent is less than an inflation rate of the pump.

4. The blood pressure measurement device of claim 1, wherein a release rate of the air through the vent is less than a release rate of the air through the valve when the valve is operating in the second state.

5. The blood pressure measurement device of claim 1, wherein:
the bladder comprises an inlet and an outlet;
the valve is coupled to the inlet; and
the vent is coupled to the outlet.

6. The blood pressure measurement device of claim 1, wherein the valve comprises the vent.

7. The blood pressure measurement device of claim 1, wherein the bladder comprises the vent.

8. A blood pressure measurement device comprising:
a cuff configured to wrap around a limb of a user;
a bladder coupled to the cuff and configured to compress the limb when inflated;
a pump operative to inflate the bladder; and
a valve comprising:
a housing;
a diaphragm contained within the housing and operative to:
in a first state, allow the pump to inflate the bladder; and
in a second state, release air from the bladder at a first rate; and
a vent configured to continuously release the air from the bladder at a second rate that is less than the first rate.

9. The blood pressure measurement device of claim 8, wherein:
the housing defines an inlet coupled to the pump, a first outlet coupled to the bladder, and a second outlet open to an external environment;
in the first state, the diaphragm allows air to move from the inlet to the first outlet and prevents air from releasing from the second outlet; and
in the second state, the diaphragm allows air to release from the second outlet.

10. The blood pressure measurement device of claim 8, wherein the vent comprises an opening within the housing that couples the bladder to an external environment.

11. The blood pressure measurement device of claim 8, wherein the vent comprises an air permeable membrane coupled to the housing.

12. The blood pressure measurement device of claim 11, wherein the air permeable membrane comprises a polytetrafluorethylene material.

13. The blood pressure measurement device of claim 8, wherein:
the pump inflates the bladder at a third rate; and
the second rate is less than the third rate.

14. The blood pressure measurement device of claim 8, wherein:
the diaphragm comprises an air impermeable material; and
the vent comprises an air permeable material.

15. The blood pressure measurement device of claim 8, wherein the vent is positioned within an outer wall of the housing and provides an air release path between an interior volume of the bladder and an external environment.

16. A blood pressure measurement device comprising:
a cuff configured to wrap around a limb of a user;
a bladder coupled to the cuff and configured to compress the limb when inflated;
a pump operative to inflate the bladder; and
a valve comprising:
a housing defining an outlet that couples the bladder to an external environment; and
a diaphragm contained within the housing, comprising an air permeable material, and operative to:
in a first state, block the outlet and allow air to vent through the air permeable material; and
in a second state, allow the air in the bladder to vent from the outlet.

17. The blood pressure measurement device of claim 16, wherein:
in the first state, the air passes through the air permeable material at a first rate; and
in the second state, the air passes through the outlet at a second rate, greater than the first rate.

18. The blood pressure measurement device of claim 16, wherein the diaphragm comprises:
a first portion comprising the air permeable material; and
a second portion comprising an air impermeable material.

19. The blood pressure measurement device of claim 18, wherein the first portion comprises a polytetrafluorethylene material.

20. The blood pressure measurement device of claim 16, wherein:
the air passes through the air permeable material at a first rate; and
the pump is operative to inflate the bladder at a second rate that is greater than the first rate.
